(19) United States
(12) Patent Application Publication
(10) Pub. No.: US 2013/0274858 A1
(43) Pub. Date: Oct. 17, 2013

(54) STENT WITH SHEATH AND METAL WIRE RETAINER

(71) Applicants: Eli Bar, Moshav Megadim (IL); Ofir Paz, Rishon Lezion (IL); Asher Holzer, Ra'anana (IL)

(72) Inventors: Eli Bar, Moshav Megadim (IL); Ofir Paz, Rishon Lezion (IL); Asher Holzer, Ra'anana (IL)

(73) Assignee: INSPIREMD LTD, Tel Aviv (IL)

(21) Appl. No.: 13/994,739

(22) PCT Filed: Dec. 18, 2011
(86) PCT No.: PCT/IB11/55758

§ 371 Date: Jun. 16, 2013

Related U.S. Application Data
(60) Provisional application No. 61/424,650, filed on Dec. 19, 2010.

(51) Int. Cl. A61F 2/90; B23K 11/11

(52) U.S. Cl. CPC A61F 2/90; B23K 11/11
USPC 623/1.11; 219/87

(57) ABSTRACT

A stent assembly (20) includes a stent (22) and a fiber mesh sleeve (24) covering the stent. A wire (28) runs along the stent over the sleeve and fastened to the stent at multiple points (30) so as to attach the sleeve to the stent.

### CROSS-REFERENCE TO RELATED APPLICATION

This application claims the benefit of U.S. Provisional Patent Application 61/424,650, filed Dec. 19, 2010, which is incorporated herein by reference.

### FIELD OF THE INVENTION

The present invention relates generally to implantable medical devices, and particularly to vascular implants.

### BACKGROUND OF THE INVENTION

Stents are commonly used in percutaneous coronary revascularization procedures, as well as in treating stenoses in peripheral vasculature, such as in the carotid vessels, organs and extremities. The stent is inserted through the vascular system in a contracted state, until it reaches the desired treatment location, and is then expanded in situ to press outward against the vessel wall. Stents typically comprise a mesh of large apertures, which can damage the surrounding stenotic vessel during expansion and can release dangerous emboli into the bloodstream. The damaged tissue is prone to form new scar tissue, which protrudes through the mesh of the stent and can lead to restenosis.

Some stents are fitted with a tubular, flexible jacket for preventing restenosis and reducing the risk of emboli. For example, PCT International Publication WO 2008/062414, whose disclosure is incorporated herein by reference, describes a stent assembly with a stent jacket, comprising an expandable fiber mesh structure fastened around an expandable stent. When the stent is expanded in a blood vessel, the jacket encourages formation of a stable layer of endothelial cells covering the fibers, while reducing platelet aggregation. WO 2008/062414 shows and describes a number of ways in which the stent jacket can be mounted to the stent, including both adhesive and sliding connections, made by knots.

### SUMMARY

Embodiments of the present invention that are described hereinbelow provide improved jackets for implantable devices, and particular improved methods for attachment of a jacket to a device.

There is therefore provided, in accordance with an embodiment of the present invention, a stent assembly, including a stent and a fiber mesh sleeve covering the stent. A wire runs along the stent over the sleeve and fastened to the stent at multiple points so as to attach the sleeve to the stent.

The wire may be point-welded to the stent at the multiple points. In a disclosed embodiment, the stent includes multiple struts, and the wire is fastened to the stent along an outermost strut of the stent. Typically, the wire includes first and second wires, which are fastened to the stent over the sleeve at first and second ends of the stent, respectively, without additional fastening of the sleeve to the stent between the first and second ends.

In one embodiment, the sleeve includes a knit having multiple eyes, and the points at which the wire is fastened to the sleeve are inside the eyes of the knit.

There is also provided, in accordance with an embodiment of the present invention, a method for producing a stent assembly, which includes positioning a fiber mesh sleeve over a stent. A wire is run along the stent over the sleeve and is fastened to the stent at multiple points so as to attach the sleeve to the stent.

The present invention will be more fully understood from the following detailed description of the embodiments thereof, taken together with the drawings in which:

### BRIEF DESCRIPTION OF THE DRAWING

FIG. 1 is a schematic, pictorial illustration of a stent assembly, in accordance with an embodiment of the present invention.

### DETAILED DESCRIPTION OF EMBODIMENTS

The inventors have found that for optimal performance of a stent jacket of the type described above, it is desirable that the jacket be mounted on the stent in a manner that allows the fibers of the jacket to slide over the stent as the stent expands. Embodiments of the present invention provide means and methods for such mounting.

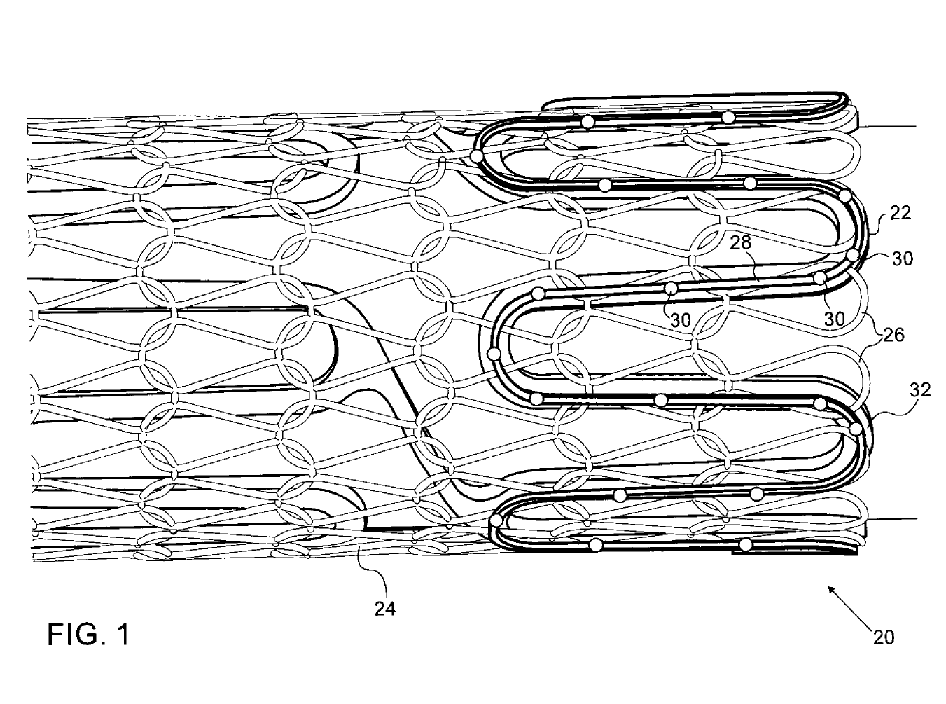

FIG. 1 is a schematic, pictorial illustration showing one end of a stent assembly 20 of this sort, in accordance with an embodiment of the present invention. Assembly 20 comprises a stent 22 made from a flexible, biocompatible material, typically a metal alloy such as stainless steel or Nitinol, as is known in the art. A fiber mesh sleeve 24 covers stent 22. The mesh in this embodiment is a knit comprising interlocking eyes 26, which define pores of a desired size when the stent assembly is expanded inside a blood vessel. It is desirable that all the eyes at the ends of sleeve 24 be fastened to stent 22 in order to prevent the knit from unraveling or sliding off the stent.

After aligning sleeve 24 with stent 22, a metal wire 28 is used to attach the sleeve to the stent. Wire 28 is typically made from a biocompatible metal, such as stainless steel, Nitinol, CoCr or any other suitable biocompatible metal alloy, and has a diameter in the range of 10-100 μm. As shown in the figure, wire 28 runs along an outermost strut 32, at the end of stent 22, over the fibers of sleeve 24. The wire is fastened to the radial surface of the underlying strut, typically by multiple point-welds 30, which may be produced by a laser micro-welder, for example. Alternatively, other methods of metal-to-metal fastening may be used. Typically, to hold the sleeve securely and prevent unraveling, one point-weld is made inside each eye 26 of the sleeve that overlies the strut. A similar welded-wire connection is made at the opposite end of the stent assembly (not shown). To allow the sleeve to slide freely over the stent during expansion of the stent, there is typically no additional fastening of the sleeve to the stent between the two ends.

Alternatively, other weld patterns may be used. For example, point-welds 30 may be more closely spaced, with two or more point-welds in at least some of eyes 26. On the other hand, the point-welds may be more widely spaced, at least in some locations, skipping over at least some of the eyes.

The point-welding technique illustrated in FIG. 1 is advantageous in that it fastens sleeve 24 to stent 22 quickly, conveniently, and securely. Between point-welds 30, however, the fibers of sleeve 24 are free to move between wire 28 and stent 22. Therefore, as stent assembly 20 expands, the fibers of sleeve 24 are able to shift relative to the stent in order to maintain relatively uniform pore sizes.

In an alternative embodiment (not shown in the figures), wire 28 runs along the axial, external edge of the outer strut of stent 22 and is point-welded to the edge, rather than to the radial surface as shown in FIG. 1. Further alternatively, other wire and weld configurations may be used and are considered to be within the scope of the present invention. For example, a wire may be placed over the sleeve and welded to the stent at the center of the stent or at some other location between the ends of the stent, in addition to or instead of the wires used at the ends of the stent as shown in the figure.

It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A stent assembly, comprising:
a stent;
a fiber mesh sleeve covering the stent; and
a wire, running along the stent over the sleeve and fastened to the stent at multiple points so as to attach the sleeve to the stent.

2. The assembly according to claim 1, wherein the wire is point-welded to the stent at the multiple points.

3. The assembly according to claim 1, wherein the stent comprises multiple struts, and wherein the wire is fastened to the stent along an outermost strut of the stent.

4. The assembly according to claim 3, wherein the wire comprises first and second wires, which are fastened to the stent over the sleeve at first and second ends of the stent, respectively, without additional fastening of the sleeve to the stent between the first and second ends.

5. The assembly according to claim 1, wherein the sleeve comprises a knit having multiple eyes, and wherein the points at which the wire is fastened to the sleeve are inside the eyes of the knit.

6. A method for producing a stent assembly, comprising:
positioning a fiber mesh sleeve over a stent;
running a wire along the stent over the sleeve; and
fastening the wire to the stent at multiple points so as to attach the sleeve to the stent.

7. The method according to claim 6, wherein fastening the wire comprises point-welding the wire to the stent at the multiple points.

8. The method according to claim 6, wherein the stent comprises multiple struts, and wherein fastening the wire comprises fixing the wire to the stent along an outermost strut of the stent.

9. The method according to claim 8, wherein fastening the wire comprises fixing first and second wires to the stent over the sleeve at first and second ends of the stent, respectively, without additional fastening of the sleeve to the stent between the first and second ends.

10. The method according to claim 6, wherein the sleeve comprises a knit having multiple eyes, and wherein the points at which the wire is fastened to the sleeve are inside the eyes of the knit.
